SEQUENCE LISTING (9 sequences)

SEQ ID NO: 1 - protein, 32 aa, Artificial Sequence
  transfection vector
KKKKKKKKKKKKKKKKRVRRGACYGLPHKFCG

SEQ ID NO: 2 - protein, 35 aa, Artificial Sequence
  transfection vector
  MOD_RES (21)..(21): epsilon amino hexanoic acid
  MOD_RES (23)..(23): epsilon amino hexanoic acid
KKKKKKKKKKKKKKKKRVRRXSXGACYGLPHKFCG

SEQ ID NO: 3 - protein, 4 aa, Artificial Sequence
  linker
RVRR

SEQ ID NO: 4 - protein, 16 aa, Artificial Sequence
  polycationic nucleic acid binding component
KKKKKKKKKKKKKKKK

SEQ ID NO: 5 - protein, 5 aa, Artificial Sequence
  linker
  MOD_RES (1)..(1): epsilon amino hexanoic acid
  MOD_RES (3)..(3): epsilon amino hexanoic acid
XSXGA

SEQ ID NO: 6 - protein, 10 aa, Artificial Sequence
  cell surface receptor binding component
CYGLPHKFCG

SEQ ID NO: 7 - protein, 7 aa, Artificial Sequence
  cell surface receptor binding component
YGLPHKF

SEQ ID NO: 8 - protein, 29 aa, Artificial Sequence
  transfection vector
KKKKKKKKKKKKKKKKRVRRGACRGDCLG

SEQ ID NO: 9 - protein, 28 aa, Artificial Sequence
  transfection vector
KKKKKKKKKKKKKKKKGACYGLPHKFCG

(19) United States
(12) Patent Application Publication
(10) Pub. No.: US 2021/0170046 A1
(43) Pub. Date: Jun. 10, 2021

(54) IMPROVED LIPID-PEPTIDE NANOCOMPLEX FORMULATION FOR MRNA DELIVERY TO CELLS

(71) Applicant: UCL Business Ltd, London (GB)

(72) Inventors: Dania Grant-Serroukh, London (GB); Stephen Lewis Hart, London (GB)

(21) Appl. No.: 16/762,886

(22) PCT Filed: Nov. 9, 2018
(86) PCT No.: PCT/GB2018/053253

§ 371 Date: May 8, 2020

(30) Foreign Application Priority Data
Nov. 10, 2017 (GB) 1718660.2

(51) Int. Cl. A61K 47/69; A61K 9/127; A61K 47/54; A61K 47/64; C12N 15/88

(52) U.S. Cl. CPC A61K 47/6911; A61K 9/1272; A61K 48/00; A61K 47/6455; C12N 15/88; A61K 47/543

(57) ABSTRACT

A liposome comprising a cationic lipid, a phospholipid and a peptide and optionally consisting of from 20 to 50% by molarity cholesterol, based on the total amount of lipids, for use in non-viral gene delivery systems, for example, in the formation of lipopolyplex transfection vectors for the delivery of mRNA to cells.

### FIELD OF THE INVENTION

The present invention relates to formulations of lipids and peptides suitable for the delivery of biologically-active materials, for example nucleic acids, especially mRNA, to a cell. The invention further relates to transfection complexes for use as non-viral vectors for the delivery of biologically-active material, such as mRNA, to cells and the use of such complexes, for example in prophylaxis, treatment and vaccination, or an in vitro laboratory setting.

### BACKGROUND TO THE INVENTION

Gene delivery for therapy or other purposes is well-known, particularly for the treatment of diseases such as cystic fibrosis and certain cancers. The term refers to the delivery into a cell of a gene or part of a gene to correct some deficiency. In the present specification the term is used also to refer to any introduction of nucleic acid material into target cells, and includes gene vaccination and the in vitro production of commercially-useful proteins in so-called cell factories.

Cell delivery systems fall into three broad classes, namely those that involve direct injection of naked DNA or RNA, those that make use of viruses or genetically modified viruses and those that make use of non-viral delivery agents. Each has its advantages and disadvantages. Although viruses as delivery agents have the advantages of high efficiency and high cell selectivity, they have the disadvantages of toxicity, production of inflammatory responses and difficulty in dealing with large DNA fragments.

Non-viral gene delivery systems are based on the compaction of genetic material into nanometric particles by electrostatic interaction between the negatively charged phosphate backbone of DNA or RNA and cationic lipids, peptides or other polymers (Dowdy, S. F., Overcoming cellular barriers for RNA therapeutics. Nat Biotechnol, 2017. 35(3): p. 222-229; Kaczmarek, J. C., P. S. Kowalski, and D. G. Anderson, Advances in the delivery of RNA therapeutics: from concept to clinical reality. Genome Med, 2017. 9(1): p. 60; Zylberberg, C., et al., Engineering liposomal nanoparticles for targeted gene therapy. Gene Ther, 2017. 24(8): p. 441-452; Oberli, M. A., et al., Lipid Nanoparticle Assisted mRNA Delivery for Potent Cancer Immunotherapy. Nano Lett, 2017. 17(3): p. 1326-1335; Cullis, P. R. and M. J. Hope, Lipid Nanoparticle Systems for Enabling Gene Therapies. Mol Ther, 2017. 25(7): p. 1467-1475; Kauffman, K. J., M. J. Webber, and D. G. Anderson, Materials for non-viral intracellular delivery of messenger RNA therapeutics. J Control Release, 2016. 240: p. 227-234; Dong, Y., et al., Poly(glycoamidoamine) Brushes Formulated Nanomaterials for Systemic siRNA and mRNA Delivery in Vivo. Nano Lett, 2016. 16(2): p. 842-8.). The use of non-viral transfection vectors that include lipids, as opposed to viruses, can result in lower toxicity, especially lower immunogenicity; greater safety; reduced cost, reasonably efficient targeting, and an enhanced packaging ability, e.g. the ability to deal with large fragments of nucleic acid material. Unfortunately, lower transfection efficiencies have been noted particularly with mRNA. Non-viral gene therapy vectors have been the subject of recent reviews: Yin H, Kanasty R L, Eltoukhy A A, Vegas A J, Dorkin J R, Anderson D G. Non-viral vectors for gene-based therapy. Nature Rev Genetics. 2014: 15:541-55; Schroeder A, Levins C G, Cortez C, Langer R, Anderson D G. Lipid-based nanotherapeutics for siRNA delivery. J Intern Med. 2010: 267:9-21; Zhao Y, Huang L. Lipid nanoparticles for gene delivery. Adv Genet. 2014: 88:13-36; Tatiparti, K., et al., siRNA Delivery Strategies: A Comprehensive Review of Recent Developments. Nanomaterials (Basel), 2017. 7(4); Riley, M. K. and W. Vermerris, Recent Advances in Nanomaterials for Gene Delivery—A Review. Nanomaterials (Basel), 2017. 7(5).

Known complexes for gene delivery include lipoplex for lipid based nucleic acid complexes, polyplex for peptide or polymer-based complexes and lipopolyplex for hybrid systems (Feigner et al., Human Gene Therapy 8, 1997, 511-512). As used herein, the term “LPD” is a form of lipopolyplex representing a formulation comprising (L) a lipid, (P) an integrin-(or other receptor-) binding peptide and (D) DNA (or other nucleic acid). LPD complexes achieve transfection via an integrin-mediated or other receptor-mediated pathway; they do not necessarily need to have an overall positive charge so undesirable serum interaction can be reduced. The peptide component provides a nucleic acid packaging function, shielding the DNA or RNA from intracellular or extracellular degradation, endosomal or otherwise. The lipid components mediate interactions with endosomal lipid bilayers by membrane fusion or permeabilisation, reducing endosomal or lysosomal degradation and allowing trafficking of the nucleic acid cargo the cytoplasm. The peptide component can be designed to be cell-type specific or cell-surface receptor specific. For example the degree of specificity for integrin or other receptors can confer a degree of cell specificity to the LPD complex. Specificity results from the targeting to the cell-surface receptors (for example integrin receptors), and transfection efficiencies comparable to some adenoviral vectors can be achieved. (Du Z, Munye M M, Tagalakis A D, Manunta M D, Hart S L. The role of the helper lipid on the DNA transfection efficiency of lipopolyplex formulations. Sci Rep. 2014: 4:7107; Welser K, Campbell F, Kudsiova L, Mohammadi A, Dawson N, Hart S L, et al. Gene delivery using ternary lipopolyplexes incorporating branched cationic peptides: the role of Peptide sequence and branching. Mol Pharm. 2013: 10:127-41; Meng Q H, Irvine S, Tagalakis A D, McAnulty R J, McEwan J R, Hart S L. Inhibition of neointimal hyperplasia in a rabbit vein graft model following non-viral transfection with human iNOS cDNA. Gene Ther. 2013: 20:979-86; Manunta M D, McAnulty R J, McDowell A, Jin J, Ridout D, Fleming J, et al. Airway deposition of nebulized gene delivery nanocomplexes monitored by radioimaging agents. Am J Respir Cell Mol Biol. 2013: 49:471-80; Kenny G D, Bienemann A S, Tagalakis A D, Pugh J A, Welser K, Campbell F, et al. Multifunctional receptor-targeted nanocomplexes for the delivery of therapeutic nucleic acids to the Brain. Biomaterials. 2013: 34:9190-200; Tagalakis A D, He L, Saraiva L, Gustafsson K T, Hart S L. Receptor-targeted liposome peptide nanocomplexes for siRNA delivery. Biomaterials. 2011: 32:6302-15; Tagalakis A D, Grosse S M, Meng Q H, Mustapa M F, Kwok A, Salehi S E, et al. Integrin-targeted nanocomplexes for tumour specific delivery and therapy by systemic administration. Biomaterials. 2011: 32:1370-6; Manunta M D, McAnulty R J, Tagalakis A D, Bottoms S E, Campbell F, Hailes H C, et al. Nebulisation of receptor-targeted nanocomplexes for gene delivery to the airway epithelium. PLoS One. 2011: 6:e26768; Grosse S M, Tagalakis A D, Mustapa M F, Elbs M, Meng Q H, Mohammadi A, et al. Tumor-specific gene transfer with receptor-mediated nanocomplexes modified by polyethylene glycol shielding and endosomally cleavable lipid and peptide linkers. FASEB J. 2010: 24:2301-13.

Peptides that target human airway epithelial cells have been reported (WO02/072616). Peptides that target dendritic cells have been reported (WO2004/108938).

Lipid/peptide vectors transfect a range of cell lines and primary cell cultures with high efficiency and low toxicity: epithelial cells (40% efficiency), vascular smooth muscle cells (50% efficiency), endothelial cells (30% efficiency) and haematopoietic cells (10% efficiency). Furthermore, in vivo transfection of bronchial epithelium of mouse has been demonstrated (Manunta M D, McAnulty R J, Tagalakis A D, Bottoms S E, Campbell F, Hailes H C, et al. Nebulisation of receptor-targeted nanocomplexes for gene delivery to the airway epithelium. PLoS One. 2011: 6:e26768; Tagalakis A D, McAnulty R J, Devaney J, Bottoms S E, Wong J B, Elbs M, et al., A receptor-targeted nanocomplex vector system optimized for respiratory gene transfer. Mol Ther. 2008: 16:907-15. Jenkins et al., Formation of LID vector complexes in water alters physicochemical properties and enhances pulmonary gene expression in vivo, Gene Therapy 2003, 10, 1026-34), rat lung (Jenkins et al. , An integrin-targeted non-viral vector for pulmonary gene therapy, Gene Therapy 2000, 7, 393-400) and pig lung (Manunta M D, McAnulty R J, McDowell A, Jin J, Ridout D, Fleming J, et al. Airway deposition of nebulized gene delivery nanocomplexes monitored by radioimaging agents. Am J Respir Cell Mol Biol. 2013: 49:471-80; Cunningham et al., Evaluation of a porcine model for pulmonary gene transfer using a novel synthetic vector, J Gene Med 2002, 4, 438-46) and with efficiency comparable to that of an adenoviral vector (Jenkins et al., 2000, as above).

A peptide for use in such LPD complexes or lipid/peptide complexes must have two functionalities: a “head group” containing a cell surface receptor—(for example integrin) recognition sequence and a “tail” that can bind DNA non-covalently. Known peptides in which these two components are covalently linked via a spacer in a way that does not interfere with their individual functions include peptides in which the “tail” is a polycationic nucleic acid-binding component, such as peptide 6 as described in WO96/15811.

Initial experiments involving LPD complexes including such peptides have indicated insufficiently high transfection properties by the systemic, or intravenous, route of delivery. The likely problem, as described for other polycationic vectors is association of the vector with serum proteins and red cell membranes leading to poor solubility and rapid clearance of the vector by the reticuloendothelial systems (Dash, P. R., Read, M. L., Barrett, L. B., Wolfert, M. A., Seymour, L. W. (1999) Gene Therapy 6, 643-50). Vectors that have displayed some transfection activity by systemic administration have been effective largely in first-pass capillary beds of organs such as the liver and lung (Fenske, D. B., MacLachlan, I., Cullis, P. R. (2001). Curr Opin Mol Ther 3, 153-8). While such non-specific transfection activity may have some therapeutic applications, safe clinical use for specific applications demands vectors with far greater target specificity.

Regarding the lipid component of the LPD complexes, cationic lipids for such a use were developed by Felgner in the late 1980 s, and reported in Proc. Natl. Acad. Sci. USA 84, 7413-7417, 1987 and in U.S. Pat. No. 5,264,618. Felgner developed the now commercially-available cationic liposome known by the trademark “Lipofectin”. The “Lipofectin” liposome is a spherical vesicle having a lipid bilayer of the cationic lipid DOTMA (2,3-dioleyloxypropyl-1-trimethyl ammonium) and the neutral phospholipid lipid DOPE (phosphatidyl ethanolamine or 1,2-dioleoyl-sn-glycero-3-phosphoethanolamine) in a 1:1 ratio.

Various other cationic liposome formulations have since been devised, most of which combine a synthetic cationic lipid and a neutral phospholipid lipid. Some, for example, are based on the glycerol-skeleton (such as DOTMA) or on cholesterol, such as DC-Chol. The aim in developing new liposome formulations has often been to optimise the delivery properties of the resulting vector for a wide variety of cell types, and for in vivo applications.

The non-viral delivery of messenger RNA (mRNA) to cells as so far been limited by the lack of an efficient vector. Attempts to deliver mRNA using known non-viral vehicles that have used for DNA or siRNA have resulted in sub-optimal levels of protein expression. Furthermore, known non-viral vehicles have poor storage stability when packaged with mRNA. Overcoming the lipid bilayer to deliver RNA into cells has remained a major obstacle for the widespread development of RNA therapeutics. Therefore, there is a need for vectors that are specifically tailored to the delivery of mRNA that deliver high levels of the mRNA to cells and lead to good levels of protein expression. There is also a need for compositions tailored to the delivery of mRNA that have good stability upon storage, in particular mRNA delivery complexes that retain their structure and functionality upon cold storage.

U.S. 2016/0038432 A1 seeks to improve lipid nanoparticle formulations for mRNA thereapy (MRT) by pre-heating a mRNA solution and/or lipid solution prior to mixing to improve encapsulation efficiency, mRNA recovery rates and partilce sizes. The lipid solution contains a cationic lipid; a helper lipid, e.g. a phopholipid; a cholesterol-based lipid e.g. cholesterol; and/or a PEG lipid. WO 2016/118725 A1 and U.S. 2017/0210698 A1 relate to lipid nanoparticle composigions to deliver mRNA comprising (i) a lipid component comprising a phospholipid, e.g. DOPE or DSPC, a PEG lipid, a structural lipid, e.g. cholesterol, new lipid compounds such as lipid KL10 having five unsatyrated alkyl chains and and optionally a cationic lipid, and (ii) mRNA. There is no disclosure in U.S. 2016/0038432 A1, WO 2016/118725 A1 or U.S. 2017/0210698 A1 of a LPD complex that comprises a peptide component.

WO 2007/138324 A2 provides a peptide derivative of formula A-B-C, wherein A is a polycationic nucleic acid-binding component, B is a spacer element peptide that is susceptible to cleavage within a cell, and C is a cell surface receptor binding component. In combination with lipid derivatives, these peptides find use in non-viral gene delivery systems. WO 2007/138324 A2 discloses a liposome comprising DOTMA ((2,3-dioleyloxypropyl-1-trimentyl ammonium) and DOPE (phosphatidyl ethanolamine or 1,2-dioleoyl-sn-glycero-3-phosphoethanolamine) in combination with the peptide sequence K₁₆RVRRGACYGLPHKFCG (SEQ ID NO: 2), for use in non-viral gene delivery. FIG. 2 of WO 2007/138324 A2 presents the results of transfection, using said liposome, of mouse neuroblastoma cells, mouse endothelial cells, and human bronchial epithelial cells.

### SUMMARY OF THE INVENTION

In a first aspect, the present invention provides a liposome for the non-viral delivery of a nucleic acid to a cell, comprising a cationic lipid, a phospholipid and a peptide and, optionally, cholesterol, wherein:
a) the cationic lipid is selected from DTDTMA (ditetradecyl trimethyl ammonium), DHDTMA (dihexadecyl trimethyl ammonium) or DOTMA (2,3-dioleyloxypropyl-1-trimentyl ammonium); and
b) the phospholipid is DOPE (phosphatidyl ethanolamine or 1,2-dioleoyl-sn-glycero-3-phosphoethanolamine); and
c) the peptide has the amino acid sequence K16RVRRXSXGACYGLPHKFCG (SEQ ID NO: 2),

or wherein:
a) the cationic lipid is DTDTMA (ditetradecyl trimethyl ammonium); and
b) the phospholipid is DOPC (phosphatidyl choline or 1,2-dioleoyl-sn-glycero-3-phosphoethanoltrimethylamine); and
c) the peptide has the amino acid sequence K₁₆RVRRGACYGLPHKFCG (SEQ ID NO: 1) or K₁₆RVRRXSXGACYGLPHKFCG (SEQ ID: 2).

Thus, the present invention provides a liposome for the non-viral delivery of a nucleic acid to a cell, comprising: (i) DTDTMA, DOPE and K₁₆-RVRR-XSXGA-CYGLPHKFCG (SEQ ID NO: 2); (ii) DOTMA, DOPE and K₁₆-RVRR-XSXGA-CYGLPHKFCG (SEQ ID NO:2); (iii) DTDTMA, DOPC and K₁₆-RVRR-XSXGA-CYGLPHKFCG (SEQ ID NO: 2); (iv) DTDTMA, DOPC and K₁₆-RVRR-GA-CYGLPHKFCG (SEQ ID NO: 1); or (v) DHDTMA, DOPE and K₁₆-RVRR-XSXGA-CYGLPHKFCG (SEQ ID NO: 2), and also optionally, comprising cholesterol.

It has surprisingly been found that the liposomes of the first aspect of the invention have a significantly improved transfection efficacy compared to known liposomes, such as the LPD complexes described in the publications discussed above in the section headed “Background to the invention”. For example, it was demonstrated that transfection vectors formulated with liposomes of the invention had substantially increased luciferase expression in B16 F10 cells than similar formulations comprising alternative combinations of cationic lipid, phospholipid and peptide.

In a second aspect, the invention provides a liposome for the non-viral delivery of a nucleic acid to a cell, comprising the cationic lipid, phospholipid and peptide as defined for the first aspect of the invention and consisting of from 20 to 50% by molarity cholesterol, based on the amount of total lipids (i.e. cationic lipids, phospholipids and cholesterol). A liposome of the first aspect of the invention may optionally consist of from 20 to 50% by molarity cholesterol, based on the amount of total lipids (i.e. cationic lipids, phospholipids and cholesterol). It has been found that incorporation of substantial amounts of cholesterol in liposome formulations, especially those of the first aspect of the invention, improves protein expression.

In a third aspect, the invention provides a transfection complex comprising the liposome of the first or second aspect of the invention and a nucleic acid. The nucleic acid is advantageously RNA, especially mRNA.

In a fourth aspect, the invention provides a pharmaceutical composition which comprises the liposome of the first or second aspect of the invention or the transfection complex of the third aspect of the invention in admixture or conjunction with a pharmaceutically suitable carrier.

In a fifth aspect, the invention provides a liposome of the first or second aspect of the invention or a transfection complex of the third aspect of the invention for use in therapy.

In a sixth aspect, the invention provides a method for the treatment or prophylaxis of a condition caused in a human or in a non-human animal by a defect and/or a deficiency in a gene or for therapeutic or prophylactic immunisation, or for anti-sense or RNAi therapy, which comprises administering the liposome of the first or second aspect of the invention or a transfection complex of the third aspect of the invention to the human or to the non-human animal.

In a seventh aspect, the invention provides a method for the treatment of a human or non-human animal suffering from a cancer which comprises administering the liposome of the first or second aspect of the invention or a transfection complex of the third aspect of the invention to the human or to the non-human animal.

In an eighth aspect, the invention provides the use of the liposome of the first or second aspect of the invention or a transfection complex of the third aspect of the invention for the manufacture of a medicament for the treatment or prophylaxis of a condition caused in a human or a non-human animal by a defect and/or a deficiency in a gene, or for therapeutic or prophylactic immunisation, or for mRNA therapy, or for the treatment or prophylaxis of cancer in a human or a non-human animal.

In a ninth aspect, the invention provides liposome of the first or second aspect of the invention or a transfection complex of the third aspect of the invention for use in the treatment or prophylaxis of a condition caused in a human or a non-human animal by a defect and/or a deficiency in a gene, or for therapeutic or prophylactic immunisation, or for RNA therapy.

In a tenth aspect, the invention provides the liposome of the first or second aspect of the invention or a transfection complex of the third aspect of the invention for use in the treatment or prophylaxis of cancer in a human or a non-human animal.

### BRIEF DESCRIPTION OF THE FIGURES

FIG. 1a: graph showing Luciferase expression assay results with 15 different lipid-peptide-mRNA particles (RLPs)

FIG. 1b: graph showing Luciferase expression assay results with 44 different lipid-peptide-mRNA particles (RLPs)

FIG. 2: graph showing cholesterol effect on transfection efficiency with C14-DOPE-35

FIG. 3: graph showing cholesterol effect on transfection efficiency with C18-DOPE-35

FIG. 4: graph showing cholesterol effect on transfection efficiency with C14-DOPC-32

FIG. 5: graph showing cholesterol effect on transfection efficiency with C14-DOPC-35

FIG. 6: graph showing cholesterol effect on transfection efficiency with C16-DOPE-35

FIG. 7: graph showing Luciferase expression at different cholesterol molarities with C11-DOPE-27

FIG. 8: graph showing effect of varying the mRNA:lipid:peptide ratio on transfection efficiency with C14 DOPE 35 with 30% by molarity cholesterol

FIGS. 9a, 9b and 9c: graphs showing transfection efficiency in cell lines other than B16 F10 with C14 DOPE 35, with or without 30% by molarity cholesterol

FIG. 10: graph showing cholesterol effect on in vivo transfection efficiency with C14 DOPE 35

### DETAILED DESCRIPTION OF THE INVENTION

### Cationic Lipids (a)

The cationic lipid may be DTDTMA (ditetradecyl trimethyl ammonium), DOTMA (2,3-dioleyloxypropyl-1-trimentyl ammonium) or DHDTMA (dihexadecyl trimethyl ammonium). In addition to the cation, the cationic lipids may comprise a counter anion, for example, an inorganic counter ion, especially a pharmaceutically acceptable anion such as chloride or bromide.

Cationic lipids comprising the above cations and chloride counter anions are illustrated below:

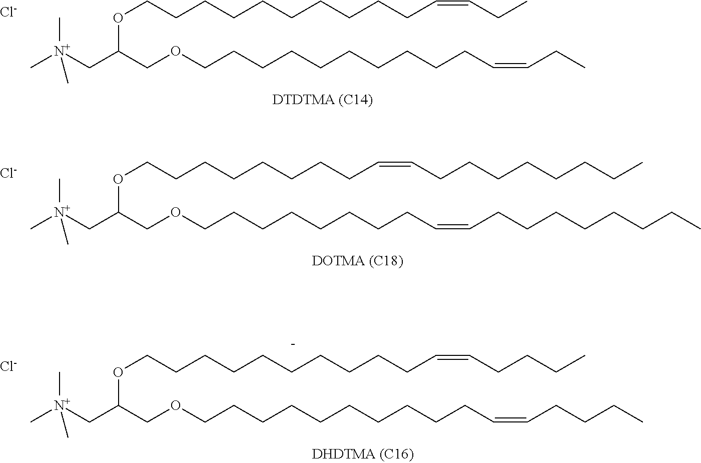

### Phospholipid (b)

The term “phospholipid” refers to a lipid comprising a fatty acid chain and a phosphate group. Phospholipids are typically neutral molecules in that they do not have an overall charge, unlike a cationic lipid which is positively charged. Phospholipids are typically zwitterionic compounds comprising both positive and negatively charged components, but no overall charge. As such, phospholipids are a typically classified as neutral lipids.

The phospholipid may be DOPE (phosphatidyl ethanolamine or 1,2-dioleoyl-sn-glycero-3-phosphoethanolamine) or DOPC (phosphatidyl choline or 1,2-dioleoyl-sn-glycero-3-phosphoethanoltrimethylamine) as illustrated below. In a preferred embodiment, the phospholipid is DOPE. DOPE is partially unsaturated and has been found to adopt a conical shape.

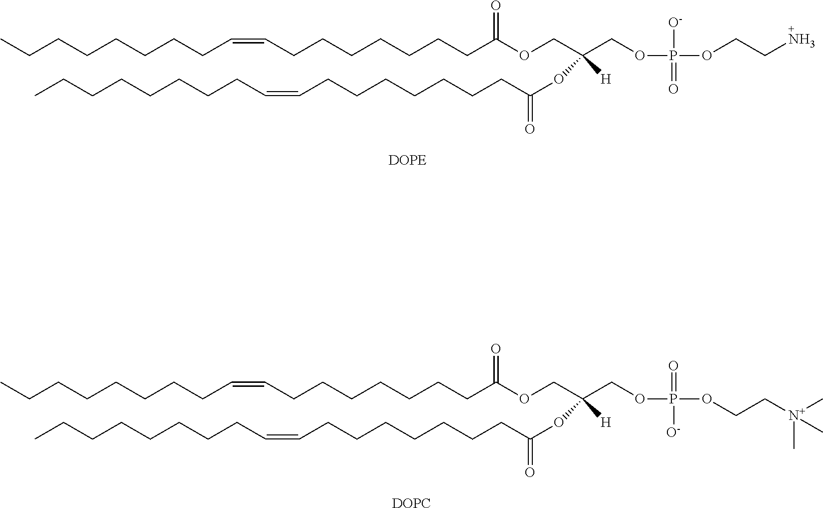 [SMILES: CCCCCCCC/C=C\CCCCCCCC(=O)OC[C@H](COP(=O)([O-])OCCN)OC(=O)CCCCCCC/C=C\CCCCCCCC ; CCCCCCCC/C=C\CCCCCCCC(=O)OC[C@H](COP(=O)([O-])OCC[N+](C)(C)C)OC(=O)CCCCCCC/C=C\CCCCCCCC ; "DOPC"]

### Peptides (c)

The peptide is of the structure A-B-C wherein:
A is a polycationic nucleic acid-binding component,
B is a spacer element comprising the amino acid sequence RVRR (SEQ ID NO: 3), and
C is a cell surface receptor binding component.

The term “polycationic nucleic acid-binding component” is well known in the art and refers to polymers having at least 3 repeat cationic amino acid residues or other cationic unit bearing positively charged groups, such polymers being capable of complexion with a nucleic acid under physiological conditions. An example of a nucleic acid-binding polycationic molecule is an oligopeptide comprising one or more cationic amino acids. Such an oligopeptide may, for example, be an oligo-lysine molecule, an oligo-histidine molecule, an oligo-arginine molecule, an oligo-ornithine molecule, an oligo diaminopropionic acid molecule, or an oligo-diaminobutyric acid molecule, or a combined oligomer comprising or consisting of any combination of histidine, arginine, lysine, ornithine diaminopropionic acid, and diaminobutyric acid residues. Any of the above oligopeptides may have, for example, a total of from 3 to 35, for example, from 5 to 25 residues, preferably from 10 to 20 residues, for example, from 14 to 18 residues, for example 16 residues.

The polycationic nucleic acid-binding component typically comprises anoligolysine having from 3 to 35, for example, from 2 to 25, for example, form 10 to 20 lysine residues, for example, from 13 to 19, for example, from 14 to 18, for example, from 15 to 17 residues, for example, 16 residues i.e. [K]₁₆ (SEQ ID NO: 4), “K” denoting lysine.

Other polycationic nucleic acid-binding components that are biologically equivalent to oligolysines, especially equivalent to K₁₆, may be used in the liposome of the invention.

Further examples of polycationic components include dendrimers and polyethylenimine. Polyethylenimine (PEI) is a non-toxic, cross-linked cationic polymer with gene delivery potential (Proc. Natl. Acad. Sci., 1995, 92, 7297-7301). Polyethylenimine is obtainable from Fluka (800 kDa) or from Sigma (50 kDa) or alternatively pre-diluted for transfection purposes from PolyPlus-tranfection (Illkirch, France). Typically, PEI is most efficient when used in a 9 fold excess over DNA, the excess ratio being calculated as PEI nitrogen: DNA phosphate, and at pH 5 to 8. Such parameters may be optimised in a manner familiar to the person skilled in the art.

The spacer element peptide B advantageously includes a cleavable portion that is susceptible to cleavage within a cell. A spacer element peptide B that includes a cleavable portion that is susceptible to cleavage within a cell may be susceptible to cleavage within the endosome, lysosome, and/or cytoplasm of a cell. Susceptible to cleavage is understood herein to mean that the element is susceptible to cleavage over a timescale during which the components A and C remain intact. Element B is cleaved more rapidly than the cellular peptide-degradation pathways take effect. The cleavable portion typically includes from 3 to 6 amino acids, for example 4 amino acids. The spacer element B includes the amino acid sequence RVRR (SEQ ID NO:3) as the cleavable portion. The amino acid sequence RVRR (SEQ ID NO:3 is susceptible to enzymatic cleavage by the endosomal protease furin. Advantageously, the cleavable portion of the spacer element peptide B is bonded to the nucleic acid-binding component A.

The spacer element peptide B additionally comprises a linker. selected from XSXGA (SEQ ID NO:5) or GA. The linker is at the end of the spacer element peptide B which is bonded to the cell surface receptor binding component C.

The spacer element peptide B comprises a cleavable portion comprising the sequences RVRR (SEQ ID NO: 3), GA and optionally XSX, which is bonded to the nucleic acid-binding component A and a linker which is bonded to the cell surface receptor binding component C.

The cell surface receptor binding component C comprises a peptide. The cell surface receptor binding component C comprises a receptor binding portion which comprises an amino acid sequence that binds to cell surface receptors. The cell surface receptor binding component C advantageously comprises a receptor binding portion which is capable of binding to human airway epithelial (HAE) cells. Examples of HAE cell-binding peptides are described in WO 02/072616.

The cell surface receptor binding component C comprises a peptide comprising a cyclic region. Cyclic peptides is formed by the provision of at least two cysteine residues in the peptide, thus enabling the formation of a disulphide bond. Accordingly, cell surface receptor binding components C consist of or comprise a peptide having two or more cysteine residues that are capable of forming one or more disulphide bond(s). The cysteine residues flank the primary receptor binding portion.

The cell surface receptor binding component C comprises the amino acid sequence CYGLPHKFCG (SEQ ID NO: 6).

The peptide of structure A-B-C comprises a nucleic acid binding polycation, such as polylysine, bonded to RVRR (SEQ ID NO: 3), the cleavable portion of a spacer element, followed by a linker portion comprising the amino acid sequence XSXGA (SEQ ID NO: 5) or GA bonded to a cell surface receptor binding component YGLPHKF (SEQ ID NO: 7), flanked by two cysteine residues.

The peptide of the present invention is selected from

Peptide 35:
(SEQ ID NO: 2)
K₁₆-RVRR-XSXGA-CYGLPHKFCG;
and
Peptide 32:
(SEQ ID NO: 1)
K₁₆-RVRR-GA-CYGLPHKFCG.

Other peptide sequences that are variants or derivatives of peptide 32 or peptide 35, and which are biologically equivalent to either of these peptides, may alternatively be included in the liposome of the present invention.

Liposomes

The invention provides the use of a transfection complex of the third aspect of the invention in the formation of a lipopolyplex (LPD) transfection vector. The transfection vector may be used to target an entity to a cell, the entity being a nucleic acid, preferably mRNA, or another molecule, for example, a therapeutically or pharmaceutically active molecule, or a molecule comprising a detectable label.

The liposome for the non-viral delivery of a nucleic acid to a cell, of the first aspect of the invention, comprises (i) DTDTMA, DOPE and K₁₆-RVRR-XSXGA-CYGLPHKFCG (SEQ ID NO: 2); (ii) DOTMA, DOPE and K₁₆-RVRR-XSXGA-CYGLPHKFCG (SEQ ID NO: 2); (iii) DTDTMA, DOPC and K₁₆-RVRR-XSXGA-CYGLPHKFCG (SEQ ID NO: 2); (iv) DTDTMA, DOPC and K₁₆-RVRR-GA-CYGLPHKFCG (SEQ ID NO: 1); or (v) DHDTMA, DOPE and K₁₆-RVRR-XSXGA-CYGLPHKFCG (SEQ ID NO: 2). In one embodiment, the liposome comprises (i) DTDTMA, DOPE and K₁₆-RVRR-XSXGA-CYGLPHKFCG (SEQ ID NO: 2); (ii) DOTMA, DOPE and K₁₆-RVRR-XSXGA-CYGLPHKFCG (SEQ ID NO: 2)or (v) DHDTMA, DOPE and K₁₆-RVRR-XSXGA-CYGLPHKFCG (SEQ ID NO: 2). In an especially preferred embodiment, the liposome comprises (i) DTDTMA, DOPE and K₁₆-RVRR-XSXGA-CYGLPHKFCG (SEQ ID NO: 2).

Cholesterol

In a second aspect of the invention, there is provided a liposome for the non-viral delivery of a nucleic acid to a cell, comprising the cationic lipid, a phospholipid and a peptide as defined for the first aspect of the invention, and consisting of from 20 to 50% molarity cholesterol, based on the amount of total lipids (i.e. cationic lipids, phospholipids and cholesterol). A liposome of the first aspect of the invention may optionally consist of from 20 to 50% molarity cholesterol, based on the amount of total lipids (i.e. cationic lipids, phospholipids and cholesterol). The liposomes of the invention, including those of the first and second aspects of the invention, advantageously consist of from 22 to 45% molarity cholesterol, such as from 23 to 40% molarity cholesterol, especially from 25 to 35% molarity cholesterol, based on the amount of total lipids (i.e. cationic lipids, phospholipids and cholesterol). It has been found that the transfection efficacy improves as the amount of cholesterol in the liposome formulation increases until a plateau is reached at about 30% molarity, as demonstrated in the data presented in FIG. 7.

The inclusion of cholesterol in the liposomes the first and second aspects of the invention and the transfection complexes of the third aspect of the invention has been found to improve their storage stability. Storage stability may be determined by measuring the size of liposome or transfection complexes after storage, e.g. at 4° C. for four weeks.

### Transfection Complexes

In a third aspect, the invention provides a transfection complex that comprises a liposome of the first or second aspect of the invention and (d) a nucleic acid. The transfection complex of the third aspect of the invention is typically a non-viral transfection complex, for example, LPD (or LID) complex. The nucleic acid is advantageously RNA, especially mRNA.

The ratio of components (d) nucleic acid : (a)+(b) total lipids, i.e. both cationic lipid and phospholipid combined: (c) peptide, in the transfection complex is advantageously about, 1:3:3, or 1:3:4 or 1:4:4 parts by weight. For example, (d):(a)+(b):(c) is 0.6-1.4:1.6-6.2: 1.6-6.2 parts by weight, especially 0.6-1.4:2.6-4.4:2.6-4.4 parts by weight, such as 0.6-1.4:3.6-4.4:3.6-4.4 parts by weight. Optionally, the transfection complex may consist of from 20 to 50% molarity cholesterol, advantageously from 22 to 45% molarity cholesterol, such as from 23 to 40% molarity cholesterol, especially from 25 to 35% molarity cholesterol, based on the amount of total lipids (i.e. cationic lipids, phospholipids and cholesterol). In one example, (d):(a)+(b):(c) is about 1:3:3 or 1:3:4 or 1:4:4 parts by weight and the transfection complex consists of from 20 to 50% molarity cholesterol. In another example, (d):(a)+(b):(c) is 0.6-1.4:3.6-4.4:3.6-4.4 parts by weight, and the transfection complex consists of from 25 to 35% molarity cholesterol. Advantageously, the transfection complex of the third aspect of the invention is suitable for use as a medicament or a vaccine.

Medical Uses

It has been found that a transfection complex of the third aspect of the invention improves the targeting of an mRNA-containing vector complex, e.g. to tumour cells.

The liposome of the first and second aspects of the invention thus find use in the treatment of cancer, therapeutic or prophylactic immunisation, or RNA therapy. In particular, the liposome of the first or second aspect of the invention finds use in the treatment or prophylaxis of cystic fibrosis (CF) or primary ciliary dyskinesia (PCD). The invention thus provides a method of treating cancer, therapeutic or prophylactic immunisation, or RNA therapy comprising administering a liposome of the first or second aspect of the invention in a suitable complex to the patient in an effective amount. In particular, the invention thus provides a method of treating cystic fibrosis (CF) or primary ciliary dyskinesia (PCD) comprising administering a liposome of the first or second aspect of the invention in a suitable complex to the patient in an effective amount. The transfection complexes of the third aspect of the invention thus find use in the treatment of cancers, for therapeutic or prophylactic immunisation, or for RNA therapy. In particular, the transfection complexes of the third aspect of the invention find use in the treatment of cystic fibrosis (CF) or primary ciliary dyskinesia (PCD). The invention thus provides a method of treating cancer, therapeutic or prophylactic immunisation, or RNA therapy comprising administering a transfection complex of the third aspect of the invention to the patient in an effective amount. In particular, the invention provides a method of treating cystic fibrosis (CF) or primary ciliary dyskinesia (PCD) comprising administering a transfection complex of the third aspect of the invention to the patient in an effective amount. The liposome of the first or second aspect of the invention or the transfection complex of the third aspect of the invention may be administered in a pharmaceutical composition of the fourth aspect of the invention, which comprises the liposome or transfection complex in admixture or conjunction with a pharmaceutically suitable carrier.

In a fifth aspect, the invention provides a liposome of the first or second aspect of the invention or a transfection complex of the third aspect of the invention for use in therapy. The invention further provides a liposome of the first or second aspect of the invention or a transfection complex of the third aspect invention for use as a medicament or a vaccine. For example, the fifth aspect of the invention provides a liposome of the first or second aspect of the invention or a transfection complex of the third aspect invention for use in the treatment or prophylaxis of condition caused by a defect and/or a deficiency in a gene, for use in the treatment of cancer, for therapeutic or prophylactic immunisation, or for RNA therapy. In particular, the fifth aspect of the invention provides a liposome of the first or second aspect of the invention or a transfection complex of the third aspect invention for use in the treatment or prophylaxis of cystic fibrosis (CF) or primary ciliary dyskinesia (PCD).

The nucleic acid component (d) may be any suitable nucleic acid. It may be DNA or RNA or a chemically modified nucleic acid mimetic, for example a PNA molecule. It may, for example, code for a protein that has a utility in the target cell. Advantageously, the nucleic acid is cellular messenger RNA (mRNA).

The invention also provides processes for the production of a transfection complex of the first and second aspects of the invention.

In a sixth aspect, the invention provides a method for the treatment or prophylaxis of a condition caused in a human or in a non-human animal by a defect and/or a deficiency in a gene which comprises administering a liposome of the first or second aspect of the invention or a transfection complex of the third aspect invention the human or to the non-human animal.

The term “a defect and/or a deficiency in a gene” as used herein denotes not only a defect or deficiency in the coding region of a gene, but a defect or deficiency in a control element for the gene, for example, a control element in trans or in cis, or a defect or deficiency in any other element that is involved in the transcription or translation of the gene, whether directly or indirectly.

In a seventh aspect, the invention provides a method for therapeutic or prophylactic immunisation of a human or of a non-human animal, which comprises administering the liposome of the first or second aspect of the invention together with, or the transfection complex of the third aspect invention comprising, mRNA to the human or to the non-human animal.

In an eighth aspect, the invention provides the use of the liposome of the first or second aspect of the invention or a transfection complex of the third aspect of the invention for the manufacture of a medicament for the treatment or prophylaxis of a condition caused in a human or a non-human animal by a defect and/or a deficiency in a gene, or for therapeutic or prophylactic immunisation, or for mRNA therapy, or for the treatment or prophylaxis of cancer in a human or a non-human animal.

In a ninth aspect, the invention provides liposome of the first or second aspect of the invention or a transfection complex of the third aspect of the invention for use in the treatment or prophylaxis of a condition caused in a human or a non-human animal by a defect and/or a deficiency in a gene, or for therapeutic or prophylactic immunisation, or for RNA therapy.

In a tenth aspect, the invention provides the liposome of the first or second aspect of the invention or a transfection complex of the third aspect of the invention for use in the treatment or prophylaxis of cancer in a human or a non-human animal.

### EXAMPLES

### Experimental Methods and Techniques

General Experimental Methods and Techniques

Unless otherwise noted, solvents and reagents for synthesis were reagent grade from commercial suppliers and used without further purification. Dry CH₂Cl₂ was obtained using anhydrous alumina columns using the procedure described in Pangborn, A. B.; Giardello, M. A.; Grubbs, R. H.; Rosen, R. K.; Timmers, F. J. Organometallics 1996, 15, 1518-1520. All moisture-sensitive reactions were performed under a nitrogen or argon atmosphere using oven-dried glassware. Reactions were monitored by TLC on Kieselgel 60 F₂₅₄ plates with detection by UV, potassium permanganate, and phosphomolybdic acid stains. Flash column chromatography was carried out using silica gel (particle size 40-63 μm). ¹H NMR and ¹³C NMR spectra were recorded on a Bruker AMX300 MHz, Avance-500 MHz and Avance-600 MHz machines. Coupling constants are measure in Hertz (Hz) and unless otherwise specified, spectra were acquired at 298 K. Mass spectra were recorded on Thermo Finnegan MAT 900XP, Micromass Quattro LC electrospray and VG70-SE mass spectrometers. Infrared spectra were recorded on a Shimadzu FTIR-8700 spectrometer.

In the following examples the liposomes are denoted using the abbreviation “Cnn DXXX nn”, where Cnn is the cationic lipid, e.g. C14 (DTDTMA), C18 (DOTMA) or C16 (DHDTMA); DXXX is the phospholipid, e.g. DOPE (phosphatidyl ethanolamine or 1,2-dioleoyl-sn-glycero-3-phosphoethanolamine), DOPC (phosphatidyl choline or 1,2-dioleoyl-sn-glycero-3-phosphoethanoltrimethylamine) or DSPC (1,2-distearoyl-sn-glycero-3-phosphocholine); and nn is the peptide, e.g. ME27 (27), Peptide 35 (35), Y (Y) or Peptide 32 (32).

Lipids
DTDTMA (C14), DOTMA (C18) and DHDTMA (C16) were prepared according to the method described in Hurley C A, Wong J B, Hailes H C and Tabor A B, Assymetric Synthesis of Dialkyloxy-3-alkylammonium Cationic Lipids. J. Org. Chem. 2004, 69:980-983.
DOPE is available from Avanti Polar Lipids, Alabaster, Alabama, USA.
DSPC is available from Avanti Polar Lipids, Alabaster, Alabama, USA.
DOPC is available from Avanti Polar Lipids, Alabaster, Alabama, USA.

Peptide Synthesis

The peptides described (Table IA) were synthesized using standard instruments and techniques.

TABLE IA
Peptide Sequence

| Peptide | Sequence |
|---|---|
| K₁₆ | KKKKKKKKKKKKKKKK (SEQ ID NO: 4) |
| ME27 | (K)₁₆RVRRGACRGDCLG (SEQ ID NO: 8) |
| Peptide 32 | (K)₁₆RVRRGACYGLPHKFCG (SEQ ID NO: 1) |
| Y | (K)₁₆GACYGLPHKFCG (SEQ ID NO: 9) |
| Peptide 35* | (K)₁₆RVRRXSXGACYGLPHKFCG (SEQ ID NO: 2) |

*in peptide 35 X = ε-Ahx

TABLE IB
Peptide mass

| Peptide | Mass (g · mol⁻¹) |
|---|---|
| K₁₆ | 2068 |
| ME27 | 3467.5 |
| Peptide 35 | 4184.38 |
| Y | 3303.28 |
| Peptide 32 | 3870.98 |

ME27 was synthesized on a SYRO automated peptide synthesizer.

Linear peptide sequences: The peptide was synthesized on a 20 μmol scale using 2 ml syringes with Teflon fits and 500 μl coupling volume. Fmoc-Gly preloaded NovaSyn TGT resin or Fmoc-Gly-2-Cl-Trt-resin were used for these sequences. Fmoc-Peg4-COOH was synthesized following a procedure reported previously (see synthesis of Fmoc-Haa4-COOH at page 82 of WO 2005/117985—Fmoc-Haa4-COOH was the name given to Fmoc-Peg4-COOH in that specification). The TGT resin was initially swelled for 10 min, however the 2-Cl-Trt resin needed a prolonged initial swelling time (some hours) in DMF. Routine coupling was performed with HBTU (in DMF) and DIPEA (in NMP) using a fourfold excess of reagents. Fmoc was cleaved with a 40% solution of piperidine in DMF for 3 min and a 20% solution for 10 min. Synthesis cycles consisted of 40 min coupling time, 3 min for Fmoc deprotection with 40% piperidine, another 10 min for Fmoc deprotection with 20% piperidine and washing steps. After synthesis and the last wash cycle with DMF, peptides were washed with DCM, methanol and diethyl ether (3 times each) using the “manual”/“empty” function of the Syro. Suction was applied for some more time to help evaporate the ether.

On-resin disulfide bond formation: To form disulphide bridges on resin, the resin was placed in a syringe with PE frit and swelled in DMF. After removal of excess DMF a freshly prepared solution of iodine in a minimum amount of DMF (e.g. 500 μl for a 2 ml syringe, 10 eq iodine to resin loading) was added and the syringe was vortexed during 4 h for 20 s every 4 min. The reagent solution was removed and the resin was washed 10 to 20 times with DMF, and 3 times each with DCM, methanol and ether.

Cleavage and deprotection: The syringes were transferred to the fume hood for cleavage. Cleavage was performed with a cocktail of 95% TFA, 2.5% TIS and 2.5% H₂O. A minimum amount of freshly prepared cocktail was added to cover the resin (e.g. <500 μl in the 2 ml syringes). After 4 h the cleavage solutions were passed into polypropylene (PP) tubes using a plunger and the resins were washed with another small amount of cleavage cocktail (e.g. 200 μl in the 2 ml syringes). Then the peptides were precipitated with ether (e.g. to the combined fractions of the 2 ml syringes some 4 ml of diethyl ether were added). The PP tubes were kept in the freezer for at least 15 min, then centrifuged at 3000 rpm for 3 min and solution was decanted from the peptide pellet. Centrifugation and decantation were repeated twice with about 2 ml of ether. Finally the peptides were dissolved in water or tBuOH/water (4:1) and freeze-dried. Some peptide sequences showed very poor solubility and sometimes several lyophilisation/dissolution processes with different solvent mixtures (water, tBuOH or acetonitrile) were necessary to obtain a fluffy peptide.

The peptide was analyzed by reverse phase HPLC and purified by reverse phase HPLC to >90% pure. Mass spectra were recorded using the Micromass Quattro ES-MS (Software: Masslynx) and the masses are recorded in the table IB.

Peptide 35, K16Y and Peptide 32 were purchased from AMS Bio Ltd., Birmingham, UK, and synthesised using semi-automated peptide synthesis chemistry. The peptide was analysed by reverse phase HPLC and purified where necessary by reverse phase HPLC to 85% pure. Relative molecular masses are given in the table IB.

K16 was purchased as described previously (Hart et al., Lipid-mediated enhancement of transfection by a nonviral integrin-targeting vector. Hum Gene Ther., 1998, 9, 575-585). Relative molecular masses are given in the table IB.

All these freeze-dried peptides were diluted at 10 mg/ml in water and stored at −20° C. during several months. Once thawing, aliquots of peptides are kept at 4° C. during several weeks.

mRNA

mRNA coding for firefly luciferase (CleanCap FLuc mRNA) was purchased from TriLink Biotech, San Diego, Calif. Both mRNA with no modifications and with psuedouridine modifications were used.

Nanocomplex Preparation for Transfection

The complexes were prepared to a final mRNA concentration of 2 μg/mL by adding mRNA first, followed by the liposomes, and finally the peptides at ratios of 1:3:4 respectively, unless otherwise stated. The components were mixed in OptiMEM and allowed to incubate at room temperature for 30 minutes before being used for transfection experiments.

Particle Imaging for Size and Charge Measurements

The size of RLP nanoparticles containing peptides with lipids were determined at different weight ratios of liposome to mRNA (w/w) by dynamic light scattering using a NanoZS Zetasizer (Malvern). Complexes were prepared as described above except that they were formulated in nuclease-free water rather than Opti-MEM. Samples of 1 mL (containing 1.5 μg of mRNA) were analysed to determine their size and zeta potential. The size was recorded as the average of the intensity-based distribution of particles.

Storage Stability of Liposomes or Transfection Complexes

Storage stability may be determined by measuring the size of liposome or transfection complex (RLP) particles after storage, e.g. at 4° C. for four weeks, using dynamic light scattering. The variation in particle size following storage indicates the stability of the particles. Complexes were formulated as for imaging analysis and stored either at room temperature (25° C.) or at 4° C. Samples of 1 mL (containing 1.5 μg of mRNA) were measured at specific time points over 4 weeks to determine their size using the Nano ZS (Malvern Instruments, Malvern).

Encapsulation Efficiency of mRNA within the Nanocomplexes

A Ribogreen assay (Invitrogen, Molecular Probes) was performed to determine the encapsulation efficiency of mRNA within the nanocomplexes. RLP complexes were formed as described previously except that TE buffer was used instead of water or OptiMEM. Complexes were made in 96 well plates at a 1:3:4 ratio using 100 ng mRNA per well. Samples were incubated with Ribogreen reagent and measured for fluorescence at standard fluorescein wavelengths (excitation ˜480 nm, emission ˜520 nm) using a FLUOstar Optima plate reader (BMG Labtech, Aylesbury UK). Measurements were compared to the fluorescence reading of un-complexed mRNA and the encapsulation efficiency was calculated according to the following formula:

$$\frac{( Total mRNA - Unbound mRNA )}{Total mRNA} \times 100$$

In Vitro Transfection

Cells tested included CT26 murine colon cancer cells, B16 F10 melanoma cells, and patient-derived myoblasts. Cells were seeded into 96-well plates at approximately 2×10⁴ cells per well, then incubated overnight at 37° C. in complete growth medium. The following day, RLP formulations were prepared by mixing the components in the following order: 60 μl of mRNA (R) in OptiMEM, 60 μl of liposomes (L) in OptiMEM and 80 μl of peptide (P) in OptiMEM corresponding to a weight ratio of 1:4:4, respectively. All the complexes were mixed by pipetting briefly, kept for 30 minutes at room temperature and then diluted in OptiMEM to a final volume of 1.4 ml. Two hundred microlitres of complexes corresponding at 0.1 μg of mRNA were added to each culture well after removal of the complete growth medium. All the transfections were carried out in 6 wells each. A centrifugation (1500 rpm, during 5 minutes) was performed to promote the complex sedimentation and cell contact. The cells were incubated with the complexes for 4 h at 37° C. before replacing with fresh media for 24 h, after which reporter gene expression was analyzed by luciferase assay (Promega, Madison, Wis., USA).

In Vivo Transfection

Female C57BL/6J mice were purchased from Charles River (UK). Mice were injected with 10,000 B16 F10 cells in a suspension containing 50:50 RPMI and Matrigel and left to develop tumours. Nanocomplexes were prepared as described above at an mRNA concentration of 0.2 mg/mL. 50 μL of the nanocomplex suspension was injected intra-tumourally. Control mice were not injected. 24 h following injections, the mice were culled and tumours extracted. Tumours were submerged in reporter gene assay lysis buffer, homogenised with a tissue homogeniser, and centrifuged at 14,170×g for 10 min at 4° C., after which the supernatant was removed and centrifuged for a further 10 min at 4° C. before being used in the luciferase assay.

Luciferase and Protein Assays

Cells were washed once with PBS before the addition of 20 μl of 1× Reporter Lysis Buffer (Promega, Madison, Wis., USA) to the cells for 20 min at 4° C. before freezing at −80° C. for at least 40 min, followed by thawing at room temperature. Then the luciferase activity was measured during 10 seconds using the Luciferase Assay System (Promega, Madison, Wis., USA) and an Optima Fluostar plate reader (BMG Labtech). The amount of protein present in each transfection lysate was determined with the Bio-Rad (Hercules, Calif., USA) protein assay reagent by the manufacturer's instructions, adding 20 μl from the luciferase test to 180 μl of the reagent diluted 1 in 5 and incubating at room temperature for 10 min before comparing the OD590 to a range of BSA standards. Luciferase activity was expressed as Relative Light Units (RLU) per milligram of protein (RLU/mg).

### Discussion of Results

Discussion of Results of Luciferase Expression Assay Results with Different Lipid-Peptide-mRNA Particles (RLPs)

Variations of the components of the liposome of the present invention (cationic lipids, phospholipids and peptides) were used to screen nanocomplex formulations in transfections of B16 F10 cells. Each combination of liposomes and peptide was formulated into nanocomplexes with luciferase mRNA at a constant weight ratio of 1:3:4 mRNA:liposome:peptide, and the nanocomplexes were analysed for their size, charge and % mRNA complexation.

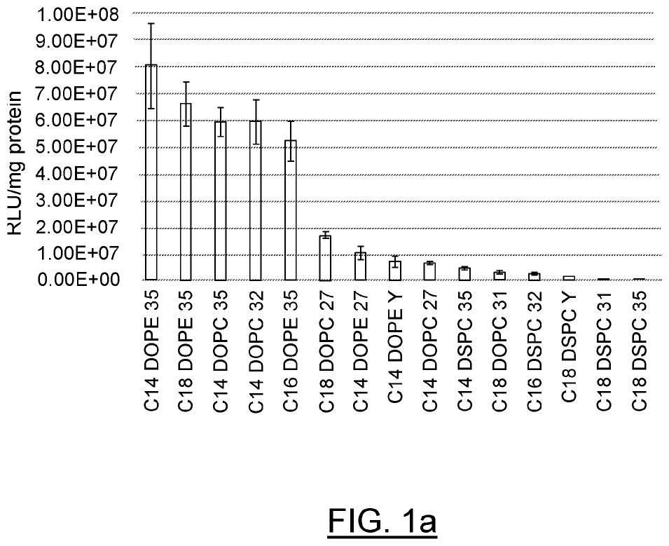

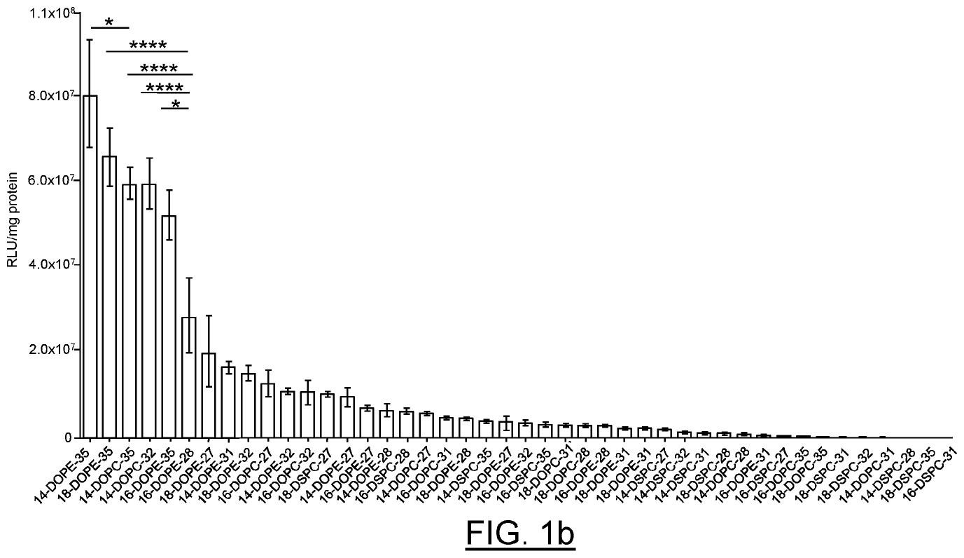

The transfection efficiency of all formulations was then compared using luciferase reporter assays. The results are reported in summary in FIG. 1a, and in greater depth in Table 1C and FIG. 1b. Formulations including both the phospholipid DOPE and peptide 35 performed better than other phospholipid/peptide combinations, with three of the top five formulations containing this mixture of components. In particular, the top five formulations performed markedly better than the prior art formulation C18 DOPE 32, disclosed in WO 2007/138324 A2 as discussed above.

The optimal formulation from this screen was C14 DOPE 35, as it produced significantly higher expression (p<0.01) than all other formulations except C18 DOPE 35. The top five formulations (C14 DOPE 35, C18 DOPE 35, C14 DOPE 35, C14 DOPE 32 and C16 DOPE 35) performed markedly better than any other formulations tested.

TABLE IC
Results of Luciferase expression assay results with
different lipid-peptide-mRNA particles (RLPs)

| RLP | RLU/mg protein |
|---|---|
| C14 DOPE 35 | 8.06E+07 |
| C18 DOPE 35 | 6.62E+07 |
| C14 DOPC 35 | 5.95E+07 |
| C14 DOPC 32 | 5.97E+07 |
| C16 DOPE 35 | 5.23E+07 |
| C16 DOPC Y | 2.82E+07 |
| C18 DOPC 27 | 1.66E+07 |
| C18 DOPE 32 | 1.98E+07 |
| C16 DOPC 27 | 1.50E+07 |
| C14 DOPE 32 | 1.26E+07 |
| C16 DOPC 32 | 1.08E+07 |
| C18 DSPC 27 | 1.07E+07 |
| C14 DOPE 27 | 1.01E+07 |
| C16 DOPE 27 | 9.68E+06 |
| C14 DOPE Y | 6.97E+06 |
| C16 DSPC Y | 6.33E+06 |
| C14 DOPC 27 | 6.10E+06 |
| C16 DOPC 31 | 5.56E+06 |
| C18 DOPE Y | 4.77E+06 |
| C14 DSPC 35 | 4.47E+06 |
| C18 DOPE 27 | 3.96E+06 |
| C16 DOPE 32 | 3.87E+06 |
| C18 DOPC 32 | 3.40E+06 |
| C16 DSPC 35 | 3.06E+06 |
| C18 DOPC 31 | 2.92E+06 |
| C18 DOPC Y | 2.80E+06 |
| C16 DOPE Y | 2.69E+06 |
| C18 DOPE 31 | 2.29E+06 |
| C16 DSPC 32 | 2.29E+06 |
| C14 DSPC 27 | 2.11E+06 |
| C14 DSPC 32 | 1.20E+06 |
| C14 DSPC 31 | 1.07E+06 |
| C18 DSPC Y | 1.06E+06 |
| C14 DOPC Y | 9.80E+05 |
| C16 DOPE 31 | 8.19E+05 |
| C16 DSPC 27 | 3.72E+05 |
| C16 DOPC 35 | 2.84E+05 |
| C18 DOPC 35 | 2.71E+05 |
| C18 DSPC 31 | 1.28E+05 |
| C18 DSPC 32 | 1.79E+05 |
| C14 DOPC 31 | 1.69E+05 |
| C14 DSPC Y | 1.40E+05 |
| C18 DSPC 35 | 3.59E+04 |
| C16 DSPC 31 | 2.47E+04 |

Peptides containing a cleavable linker (27, 32 and 35) in general performed better than those with either no linker (28) or a hydrophobic linker (31). Without wishing to be bound by theory, this suggests that rapid dissociation of the nucleic acid binding domain from the targeting moiety of the peptide once internalised is beneficial for mRNA transfection (Mustapa et al. 2009). No link between hydrocarbon chain length and either transfection efficiency or hydrodynamic diameter of the particles could be seen.

Discussion of Results of Incorporating Cholesterol into Formulations

Cholesterol was incorporated into a C14 DOPE 27 formulation and the level of luciferase expression was detected after 24 h in B16 F10 cells. The results of this experiment are shown in FIG. 7. Increasing amounts of cholesterol improved protein expression although this effect reached a plateau at 30% molarity cholesterol.

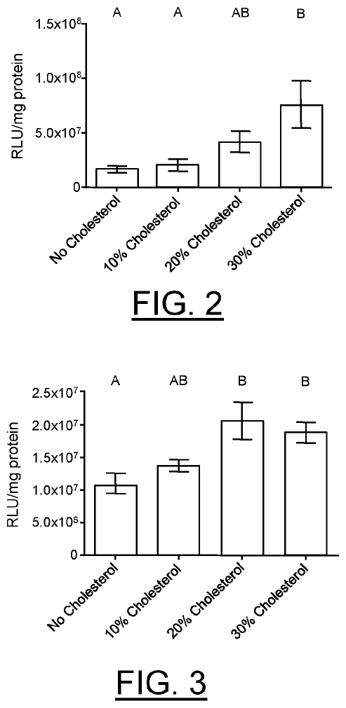

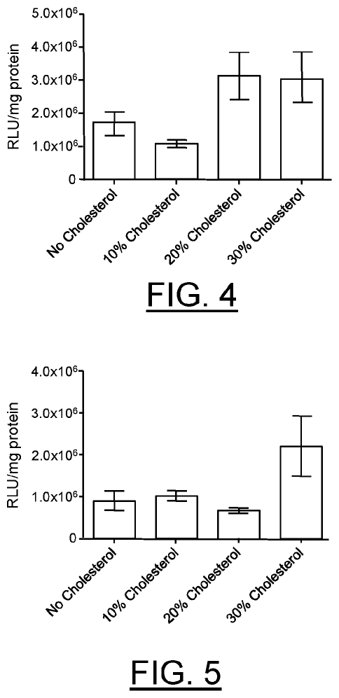

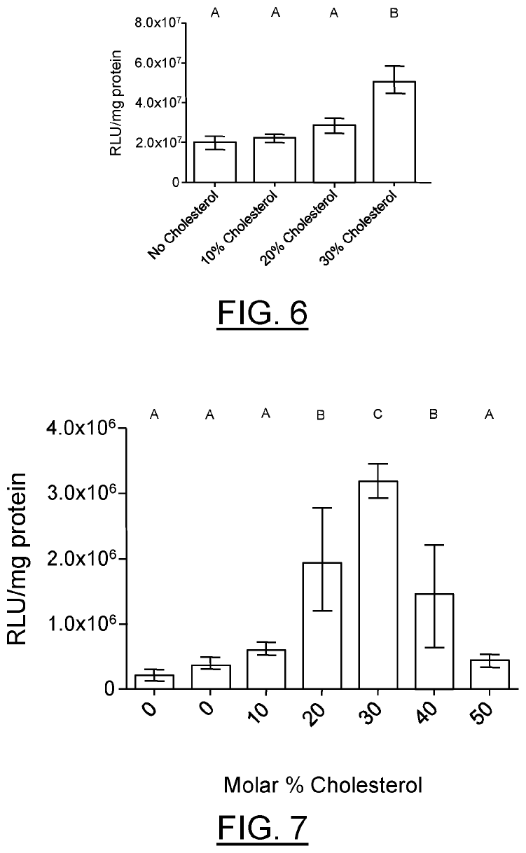

Cholesterol molar concentrations of up to 30% were tested for C14 DOPE 35, C18 DOPE 35, C14 DOPE 35, C14 DOPE 32 and C16 DOPE 35, the results of which are shown in FIG. 2, FIG. 3, FIG. 4, FIG. 5 and FIG. 6 respectively. These graphs show that the inclusion of 30% molarity cholesterol in the formulation significantly improved transfection.

Discussion of Storage Stability of Transfection Complexes

Particles of the invention were found to retain size and charge characteristics with less than 20% variation in size over a four week period of storage at 4° C. These particles also showed no change in mRNA encapsulation over this same period with ˜95% of the mRNA bound to the complexes at day 28 of storage. Whereas, particles without cholesterol steadily released mRNA over the four week period with only ˜75% of the mRNA bound to particles by day 28.

Discussion of Results of Varying the Ratios of mRNA:Lipid:Peptide

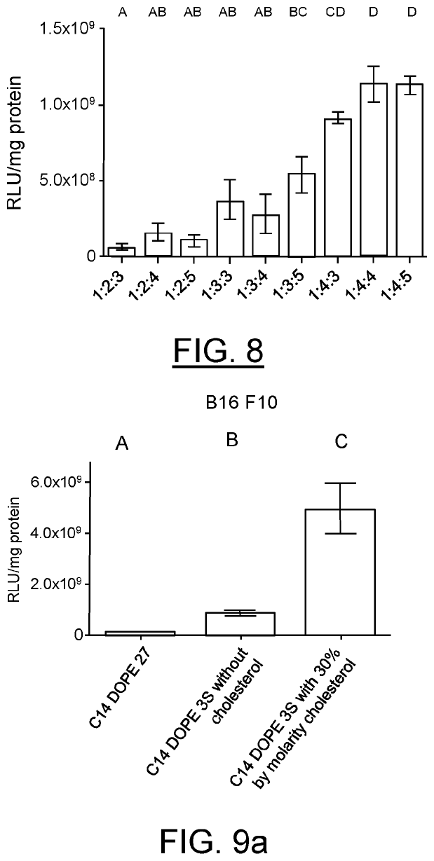

The ratio of components (d) nucleic acid : (a)+(b) total lipids, i.e. both cationic lipid and phospholipid combined: (c) peptide in the transfection complex was also investigated. Luciferase activity was measured in B16 F10 cells after transfection with the optimal formulation, C14 DOPE 35 with 30% by molarity cholesterol. Different ratios of total lipids (2-4) and peptide (3-5) were tested. The results are shown in FIG. 8, wherein formulations which share a letter are not significantly different whilst those which do not are considered significantly different (P≤0.05) based on a one-way ANOVA test. Data in FIG. 8 is presented as mean+SEM, n=6. The results shown in FIG. 8 indicate that the most effective ratio for transfection is 1:4:4. Although optimum transfection was seen at 1:4:4, a negative effect on cell proliferation was seen at higher liposome and/or peptide weight ratios. Thus, the optimal ratio was deemed to be around 1:3:4 or 1:4:4 for the five most effective formulations. For C14 DOPE 35, the optimal ratio was found to be 1:4:4.

Discussion of Results of Transfection in Other Cell Lines

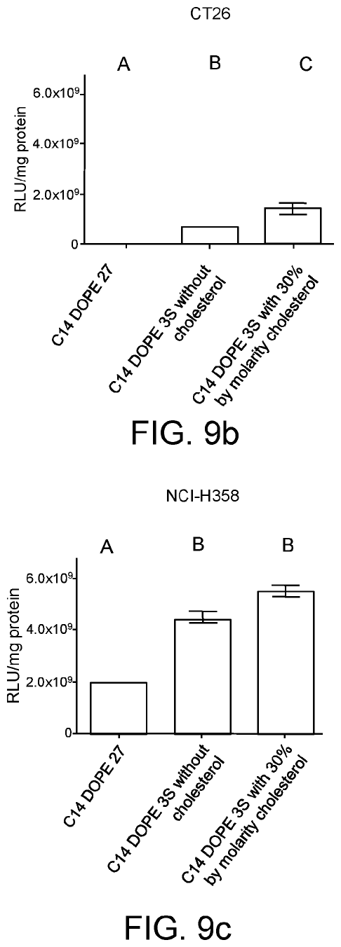

The ability of a vector to transfect multiple cell lines is a major issue in formulation development. To assess whether or not the optimal formulations (C14 DOPE 35 without cholesterol and C14 DOPE 35 with 30% by molarity cholesterol) could deliver mRNA as effectively to other cancer cells, the transfection efficiencies of these optimal formulations were compared to that of C14 DOPE 27, in two other cell lines: CT26, a mouse colon cancer, and NCI-H358, a human bronchioalveolar carcinoma cell line. The results, shown in FIGS. 9a, 9b and 9c, confirmed the trends seen in B16 F10 cells, where a significantly improved transfection was measured in cells transfected using the optimal formulations, compared to C14 DOPE 27. Although the cholesterol-containing optimal formulation (C14 DOPE 35 with 30% by molarity cholesterol) further enhanced transfection in all the cell lines tested, the degree of improvement seemed to be cell specific. The difference compared to C14 DOPE 35 without cholesterol was only statistically significant in B16 F10 and CT26 cells.

In FIGS. 9a, 9b and 9c, formulations which share a letter are not significantly different whilst those which do not are considered significantly different (P≤0.05) based on a one-way ANOVA test. Data is presented as mean+SEM, n=6.

Discussion of Results of In Vivo mRNA Delivery

Ideal mRNA delivery systems need to be able to package their cargo when exposed to proteins and endonucleases present in blood and the extracellular environment, avoiding nonspecific interactions to efficiently deliver mRNA to cells in vivo. In addition, the vector must have a good safety profile and not induce an immune response in the organism.

The optimal nanocomplex (C14 DOPE 35 with 30% by molarity cholesterol) was delivered intratumourally to B16 F10 tumours to evaluate its ability to deliver mRNA in vivo in mice. In order to assess the effect of cholesterol in vivo, C14 DOPE 35 with 30% by molarity cholesterol was compared against C14 DOPE 35 without cholesterol. Untreated tumours were used as a negative control. Tumours were harvested after 24 hours and luciferase expression quantified.

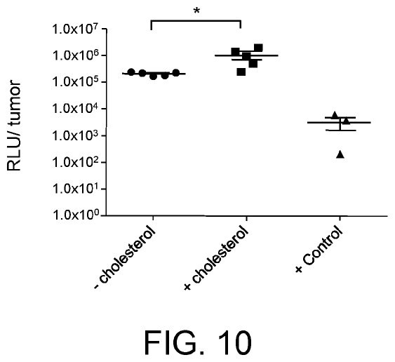

The results, shown in FIG. 10, relate to the injection of C14 DOPE 35 (both with and without cholesterol, containing 10 μg of luciferase mRNA) into B16 F10 tumours of female C57BL/6J mice, to probe their in vivo transfection efficiencies. Luciferase expression was normalised to tumour mass. The data in FIG. 10 is presented as the mean±SEM.

FIG. 10 shows that the optimal cholesterol formulation produced significantly higher levels of protein expression than the formulation lacking cholesterol. These results suggest that the lipid-peptide nanoparticles of the invention can deliver mRNA in vivo without substantial interference from serum proteins and other biological challenges.

Summary of Results

The luciferase assays described herein, the results of which are shown in Table 1C and FIGS. 1a and 1b, point to C14 DOPE 35, C18 DOPE 35, C14 DOPE 35, C14 DOPE 32 and C16 DOPE 35 as being the most effective formulations in terms of transfection efficiency. The most effective of these is C14 DOPE 35.

Moreover, the cholesterol studies described herein, the results of which are shown in FIGS. 2 to 7, suggest that the addition of 30% by molarity cholesterol to these formulations increases their transfection efficiency. The most effective cholesterol-containing formulation is C14 DOPE 35 with 30% by molarity cholesterol.

Experiments set out above (supported by FIG. 8) regarding the ratio of mRNA:lipid:peptide for C14 DOPE 35 with 30% by molarity cholesterol indicate that the optimal ratio for this formulation is 1:3:4 or 1:4:4, especially 1:4:4.

The above discussion of results in cell lines other than B16 F10 (with results shown in FIGS. 9a, 9b and 9c) when taken with the results of in vivo mRNA delivery (see FIG. 10), support the use of the invention for in vivo applications, such as in pharmaceutical compositions, in methods for the treatment or prophylaxis of a condition caused in a human or in a non-human animal, or as a medicament.

1. A composition comprising a liposome for the non-viral delivery of mRNA to a cell, the liposome comprising: (i) (a) the cationic lipid DTDTMA, (b) the phospholipid DOPE and (c) a peptide comprising the sequence K₁₆-RVRR-XSXGA-CYGLPHKFCG (SEQ ID NO: 2); (ii) (a) the cationic lipid DOTMA, (b) the phospholipid DOPE and (c) a peptide comprising the sequence K₁₆-RVRR-XSXGA-CYGLPHKFCG (SEQ ID NO: 2); (iii) the (a) cationic lipid DTDTMA, (b) the phospholipid DOPC and (c) a peptide comprising the sequence K₁₆-RVRR-XSXGA-CYGLPHKFCG (SEQ ID NO: 2); (iv) (a) the cationic lipid DTDTMA, (b) the phospholipid DOPC and (c) a peptide comprising the sequence K₁₆-RVRR-GA-CYGLPHKFCG (SEQ ID NO: 1); or (v) (a) the cationic lipid DHDTMA, (b) the phospholipid DOPE and (c) a peptide comprising the sequence K₁₆-RVRR-XSXGA-CYGLPHKFCG (SEQ ID NO: 2), and also optionally, comprising cholesterol.

2. The composition of claim 1, the liposome comprising (i) (a) DTDTMA, (b) DOPE and (c) K₁₆-RVRR-XSXGA-CYGLPHKFCG (SEQ ID NO: 2); (ii) (a) DOTMA, (b) DOPE and (c) K₁₆-RVRR-XSXGA-CYGLPHKFCG (SEQ ID NO: 2) or (v) (a) DHDTMA, (b) DOPE and (c) K₁₆-RVRR-XSXGA-CYGLPHKFCG (SEQ ID NO: 2).

3. The composition of claim 1, the liposome comprising (i) (a) DTDTMA, (b) DOPE and (c) K₁₆-RVRR-XSXGA-CYGLPHKFCG (SEQ ID NO: 2).

4. The composition of claim 1, the liposome of consisting of from 20 to 50% by molarity cholesterol, based on the total amount of lipids.

5. The composition of claim 1, the liposome consisting of from 23 to 40% by molarity cholesterol, based on the total amount of lipids.

6. The composition of claim 1, the composition comprising a non-viral transfection complex that comprises the liposome and (d) a nucleic acid, especially mRNA.

7. The composition of claim 6, wherein the non-viral transfection complex has:
from 0.6 to 1.4 parts by weight nucleic acid (d),
from 2.6 to 4.4 parts by weight total lipids (a)+(b), and
from 2.6 to 4.4 parts by weight peptide (c).

8. The composition of claim 7, wherein the ratio of components (d) nucleic acid : (a)+(b) total lipids: (c) peptide in the non-viral transfection complex is about 1:3:4 or 1:4:4 parts by weight of the non-viral transfection complex.

9. The composition of claim 6 further comprising a pharmaceutically suitable carrier.

10. A method for the treatment or prophylaxis of a condition caused in a human or in a non-human animal by a defect and/or a deficiency in a gene, or for therapeutic or prophylactic immunisation, or for RNA therapy, or for the treatment of a cancer, or for the treatment or prophylaxis cystic fibrosis (CF) or primary ciliary dyskinesia (PCD), which comprises administering a non-viral transfection complex as claimed in claim 6 to the human or to the non-human animal.

11. A The composition of claim 9 comprising an effective amount of the transfection complex for use as a medicament, for example, as a vaccine or for use in the treatment or prophylaxis of a condition caused in a human or a non-human animal by a defect and/or a deficiency in a gene, or for therapeutic or prophylactic immunisation, or for RNA therapy, or for the treatment of a cancer, or for the treatment or prophylaxis cystic fibrosis (CF) or primary ciliary dyskinesia (PCD).

12. A method for the manufacture of a medicament for the treatment or prophylaxis of a condition caused in a human or a non-human animal by a defect and/or a deficiency in a gene, or for therapeutic or prophylactic immunisation, or RNA therapy, or for the treatment of a cancer, or for the treatment or prophylaxis cystic fibrosis (CF) or primary ciliary dyskinesia (PCD), the method comprising use of a composition as claimed in claim 6.

13. The composition of claim 6, wherein the liposome comprises (i) (a) DTDTMA, (b) DOPE and (c) K16-RVRR-XSXGA-CYGLPHKFCG (SEQ ID NO: 2).

14. The composition of claim 6, wherein the liposome comprises (i) (a) DTDTMA, (b) DOPE and (c) K₁₆-RVRR-XSXGA-CYGLPHKFCG (SEQ ID NO: 2); (ii) (a) DOTMA, (b) DOPE and (c) K₁₆-RVRR-XSXGA-CYGLPHKFCG (SEQ ID NO: 2) or (v) (a) DHDTMA, (b) DOPE and (c) K₁₆-RVRR-XSXGA-CYGLPHKFCG (SEQ ID NO: 2).

15. The composition of claim 6, wherein the liposome consists of from 23 to 40% by molarity cholesterol, based on the total amount of lipids.

16. The method of claim 10, wherein the liposome comprises (i) (a) DTDTMA, (b) DOPE and (c) K₁₆-RVRR-XSXGA-CYGLPHKFCG (SEQ ID NO: 2); (ii) (a) DOTMA, (b) DOPE and (c) K₁₆-RVRR-XSXGA-CYGLPHKFCG (SEQ ID NO: 2) or (v) (a) DHDTMA, (b) DOPE and (c) K₁₆-RVRR-XSXGA-CYGLPHKFCG (SEQ ID NO: 2).

17. The method of claim 10, wherein the liposome consists of from 23 to 40% by molarity cholesterol, based on the total amount of lipids.

18. The method of claim 10, wherein the non-viral transfection complex has:
from 0.6 to 1.4 parts by weight nucleic acid (d),
from 2.6 to 4.4 parts by weight total lipids (a)+(b), and
from 2.6 to 4.4 parts by weight peptide (c).

19. The method of claim 19, wherein the ratio of components (d) nucleic acid : (a)+(b) total lipids: (c) peptide in the non-viral transfection complex is about 1:3:4 or 1:4:4 parts by weight of the non-viral transfection complex.

20. The method of claim 10, wherein the liposome comprises (i) (a) DTDTMA, (b) DOPE and (c) K16-RVRR-XSXGA-CYGLPHKFCG (SEQ ID NO: 2).
